(19) United States
(12) Patent Application Publication
(10) Pub. No.: US 2012/0283583 A1
(43) Pub. Date: Nov. 8, 2012

(54) APPARATUS AND METHOD FOR ELECTROCARDIOGRAM-ASSISTED BLOOD PRESSURE MEASUREMENT

(76) Inventors: Izmail Batkin, Gloucester (CA); Saif Ahmad, Kanata (CA); Miodrag Bolic, Nepean (CA); Voicu Zamfir Groza, Gloucester (CA); Hilmi R. Dajani, Ottawa (CA); Mohamad Forouzanfar, Ottawa (CA)

(21) Appl. No.: 13/462,670

(22) Filed: May 2, 2012

Related U.S. Application Data
(60) Provisional application No. 61/481,450, filed on May 2, 2011.

(51) Int. Cl. A61B 5/022

(52) U.S. Cl. 600/493; 600/499

(57) ABSTRACT

Apparatus, method, and software for electrocardiogram-assisted non-invasive arterial blood pressure d stiffness measurement is disclosed including brachial cuff with flexible electrodes, control box with rigid electrodes, and associated hardware/software. Cuff is wrapped around upper arm while electrodes on device are touched with fingers of other hand. Device acquires simultaneous ECG/oscillometric data during cuff deflation. Processing unit determines ECG R-peak positions to isolate arterial pulses and calculate pulse transit time. Change in pulse amplitude as function of cuff pressure is used for constructing oscillometric envelope and calculating blood pressure using empirical coefficients. Change in pulse transit time as function of cuff pressure is used independently for constructing pulse transit time envelopes and finding blood pressure with/without empirical coefficients. Fusion algorithm combines results for robust blood pressure and vessel stiffness evaluation. Device sends physiological information to personal computer/smartphone wirelessly, for further analysis. Computer/smartphone transmits information to third party for patient management.

This application claims the benefit of U.S. Provisional Patent Application No. 61/481,450, filed on May 2, 2011.

### FIELD OF THE INVENTION

The present invention relates to non-invasive automatic blood pressure measurement in humans whereby electrocardiogram (ECG) data acquisition is ergonomiclly integrated into the oscillometric blood pressure monitoring paradigm to provide robust evaluation of blood pressure and vessel stiffness.

### BACKGROUND

Accurate automatic non-invasive assessment of blood pressure employing oscillometry is a challenge. Factors like arrhythymias, obesity, and postural changes tend to obscure arterial amplitude pulsations that are sensed by the cuff, thus introducing errors in these measurements. Therefore, robust and reliable non-invasive estimation of blood pressure remains a topic of active research and inquiry.

Various prior art devices and techniques have explored newer methods that not only bolster the popular oscillometric technique but also go beyond it for estimating blood pressure. For example, the use of an ECG signal, which is a higher fidelity physiological signal, is proposed for reconstructing an oscillometric signal contaminated with artifacts to provide accurate assessment of blood pressure. Similarly, synchronized ECG signals are employed for removing motion artifacts from oscillometric signals to increase the accuracy of blood pressure measurements.

Some attempts have been made to combine blood pressure and ECG monitoring in a single device by incorporating ECG electrodes in a blood pressure cuff in an effort to render compactness to these monitors. The AMON system combines pulse and ECG sensors in a single wrist worn enclosure for continuously collecting and evaluating multi-parameter vital signs. A U.S. Patent Application proposes a combined ECG and blood pressure monitor resembling a wristwatch whereby the whole device is contained inside the wrist enclosure. Another US Patent presents a simplified ECG monitoring system in which two ECG electrodes made of sintered Ag/AgCl coating are incorporated in a brachial blood pressure cuff while a third ECG electrode (made in the same manner) is provided inside a pulse oximeter finger probe. A widely researched method that goes beyond oscillometry comprises the estimation of blood pressure from pulse transit time or pulse velocity—the time taken by a cardiac pulse to travel between the heart and a peripheral arterial site or between two peripheral arterial sites. Many prior art publications propose the pulse transit time-blood pressure correlation analysis method for assessing blood pressure. Here, the inverse correlation between pulse transit time and blood pressure is utilized for blood pressure estimation, whereby a rise in blood pressure causes the pulse transit time to decrease and vice versa. Other researchers have proposed to estimate blood pressure by studying the dependence of pulse transit time on applied cuff pressure.

All of the above described techniques and methods show promise towards increasing the robustness of automatic non-invasive blood pressure measurement. However, they have not been suitably integrated into one system along with appropriate analysis algorithms. The methods that propose to increase accuracy of oscillometric blood pressure measurement by analyzing it in the presence of a higher fidelity ECG signal employ obtrusive gel chest and/or auxiliary electrodes and do not obtain blood pressure information from the dependence of pulse transit time on cuff pressure. The systems that propose integrating ECG monitoring inside a blood pressure monitor do not report any analysis algorithms that may be employed to increase accuracy of blood pressure measurement. In addition, these systems employ hard and/or gel electrodes under cuff, which are ergonomically problematic and may affect arterial pulsations sensed by the cuff. The pulse transit time-blood pressure correlation method is cumbersome since it requires frequent calibration using another blood pressure monitor. Moreover, since blood pressure-pulse transit time correlations are weak, this method is not reliable for robust blood pressure estimation. Finally, the approaches that measure blood pressure from the dependence of pulse transit time on cuff pressure use a number of auxiliary pressure and/or ECG sensors to the cuff rendering them inconvenient and do not obtain blood pressure information from the oscillometric signal itself.

### SUMMARY OF THE INVENTION

The present invention addresses the above-mentioned limitations in the field of non-invasive automatic blood pressure measurement. An ECG-assisted blood pressure monitoring device is described wherein high fidelity ECG data acquisition is ergonomically integrated with the oscillometric blood pressure monitoring paradigm and a comprehensive analysis platform is provided for robust blood pressure and vessel stiffness evaluation. Dry, thin, and flexible ECG electrodes are incorporated on the inner surface of a brachial blood pressure cuff. In addition, dry and rigid ECG electrodes are provided on the control unit. The control unit includes hardware and software for simultaneous ECG and arterial pulse wave or oscillometric data acquisition and analysis. During a measurement, the cuff is wrapped around the upper arm while electrodes on the control unit are touched with the other hand. All measurements are accomplished by inflating the cuff to a pressure above the expected systolic pressure, and then, deflating at a desired constant rate (generally, about 3 mmHg/s) until a pressure pressure of less than the expected diastolic pressure is reached. At this point, the residual pressure inside the cuff is completely released and the measurement complete.

In one embodiment of the invention, two flexible ECG electrodes made of conductive fabric are stitched on the inner side of a brachial blood pressure cuff which has an inflatable bladder inside. One of the conductive fabric electrodes acts as the first ground electrode while the other acts as the first sensing electrode for ECG data harvest. Both these electrodes are dry and re-usable ECG electrodes. The large surface area of these electrodes and their soft texture ensures that they make good and permanent contact with the skin to enable acquisition of a high quality ECG signal. Moreover, the softness and flexibility of these ECG electrodes ensures that they do not affect the pressure sensing capability and accuracy of the blood pressure cuff.

Two rigid ECG electrodes, one made of stainless steel and the other made of high-impedance rubber, are attached on the device box. The stainless steel electrode acts as the second ground electrode while the high-impedance rubber electrode acts as the second sensing electrode for ECG data harvest. Again, these are dry and re-usable ECG electrodes. Both the flexible electrode pair inside cuff and the rigid electrode pair on device box are designed as active ECG electrodes whereby the respective amplification circuits are in close proximity with the electrodes. That is, the ECG signal is amplified at a site physically very close to the electrodes and then transmitted further. This reduces the problem of signal transmission loss and interference in the signal transmission line. As a result, the acquired ECG signal is of high quality with minimum noise.

Moreover, the use of a high-impedance sensing electrode on the device box reduces the problem of half-cell potential to allow rapid and good quality ECG data acquisition. Rapid ECG data acquisition is an important requirement in an oscillometric blood pressure monitor. This is because an oscillometric signal acquired by such a monitor generally has a duration range of 30-90 s. Therefore, the response of the ECG system should be fast enough to enable acquisition of a good quality analogous ECG signal in the same timeframe.

The two active ECG electrode pairs (two conductive fabric electrodes along with amplification circuitry inside cuff, and one high-impedance rubber and one stainless steel electrode along with amplification circuitry on device box) are connected to an electrical conditioning unit for further amplification and filtering. The acquired ECG signal is similar to the one obtained using a lead 1 configuration. The ECG measuring unit has the capability of injecting a high frequency (˜20 KHz) and low magnitude current (˜100 μA) into the ECG measuring circuit for checking the goodness of contact between the electrodes and the human body. The device generates an alarm alerting the user in case the contact between the electrodes and the human body is found to be weak or inappropriate.

A motorized pump inflates the cuff while a pressure transducer measures cuff pressure. A voltage-controlled pressure release valve guided by ECG R-peak information accomplishes cuff deflation during which the monitor acquires analogous arterial puke wave or oscillometric data.

ECG R-peak locations are used for isolating arterial pukes that facilitates the calculation of their amplitude. Moreover, ECG R-peak locations and arterial pukes are used for calculating pulse transit time.

An oscillometric envelope is constructed by mapping the change in arterial pulse amplitude in response to changing cuff pressure. The cuff pressure at which the maximum of the oscillometric envelope is reached gives the mean blood pressure. Empirical coefficients, that is, certain ratios of the maximum of the oscillometric envelope, are used for evaluating diastolic and systolic blood pressure.

Similarly, pulse transit time envelopes are constructed by mapping the change in pulse transit time, measured between ECG R-peak and different locations on the arterial pulse, in response to changing cuff pressure.

The cuff pressure at which the maximum of the pulse transit time envelope, calculated from ECG R-peak and maximum slope of arterial pulse wave, is reached gives the mean blood pressure. Empirical coefficients, that is, certain ratios of he maximum of the pulse transit time envelope, calculated from ECG R-peak and maximum slope of arterial pulse wave, are used for evaluating diastolic and systolic blood pressure.

Additionally, the cuff pressure at which the maximum of the pulse transit time envelope, calculated from ECG R-peak and top of arterial pulse wave, is reached gives the systolic blood pressure. Finally, the cuff pressure at which the maximum of the pulse transit time envelope, calculated from ECG R-peak and bottom of arterial pulse wave, is reached gives the diastolic blood pressure. We note that no empirical coefficients are required for calculating diastolic and systolic blood pressure when puke transit time envelopes are calculated from ECG R-peak and top/bottom of arterial pulse wave.

A fusion algorithm is employed for combining the blood pressure information obtained from the ECG-assisted oscillometric and pulse transit time-cuff pressure analyses to provide a robust and accurate evaluation of blood pressure.

Finally, a regression analysis is carried out between the oscillometric and pulse transit time envelopes to provide vessel stiffness parameters.

The device has capability of repeating blood pressure measurements periodically, for continuous blood pressure monitoring.

A central processing unit (CPU) runs all software and interacts with various device components to simultaneously acquire/analyze ECG and oscillometric data, and to transmit information as required. The device has onboard memory to store all information and a liquid crystal display, which displays the measured blood pressure values as well as the ECG and the arterial pulse waveforms. Moreover, the device has functionality of transmitting information to a personal computer and/or a smartphone wirelessly.

The personal computer and smartphone have customized software for storing, analyzing, and visualizing physiological information received from the device. This allows the user to assess/visualize parameters such as blood pressure trends, arterial stiffness variations, and arrhythmia periods in a flexible and adjustable manner.

Once physiological information is stored inside the personal computer and/or smartphone, it is transmitted via Internet or cellular network to designated recipients for medical evaluation and patient management.

The invention describes a sensing unit, comprising a cuff for measuring blood pressure, a first dry flexible sensing electrode positioned between a body part and an inside surface of the cuff, for connection to a human body. One or more dry flexible ground electrodes is positioned between a body part and an inside surface of the cuff for connection to the human body and a second sensing dry electrode is provided for connection to the human body such that a heart of the human body is intermediate the first sensing and second sensing electrodes.

The system further comprises a second dry ground electrode near the second sensing electrode, for equalizing static potential on body and reducing noise.

The first and second sensing electrodes are active electrodes to reduce transmission noise. Moreover, the first and second sensing electrodes are high impedance electrodes to reduce half-cell potential. Further, the system comprises a device box, wherein the second sensing electrode is positioned.

The system for non-invasive blood pressure estimation comprises an electrocardiogram (ECG) measuring unit, an arterial pulse wave measuring unit in communication with the ECG measuring unit, a cuff for measuring blood pressure in communication with the arterial pulse wave measuring unit, two or more electrodes connected to the ECG measuring unit, and an analysis unit connected to the ECG and arterial pulse wave measuring unit.

The analysis unit comprises an ECG measuring subunit, a cuff pressure and arterial pulse wave measuring subunit, a subunit that uses ECG R-peak information for isolating arterial pulse waves, a subunit for measuring puke transit time between ECG R-peak and specific points on the arterial pulse wave and mapping the measured pulse transit time with corresponding cuff pressure, obtaining pulse transit time envelopes, a subunit for de-trending cuff pressure signal and finding arterial pulse wave amplitude using ECG R-peak information, and mapping the measured amplitude with corresponding cuff pressure, obtaining an oscillometric envelope. A subunit analyzes morphology of pulse transit time envelopes, to obtain pulse transit time-based blood pressure estimation. Moreover, a subunit analyzes morphology of the oscillometric envelope, to obtain oscillometric blood pressure estimation.

In this system, the ECG measuring unit uses a high frequency, low magnitude current for checking the quality of contact between the electrodes and the human body.

Moreover, the ECG measuring unit comprises two or more flexible dry electrodes attached to the internal surface of a brachial cuff and two or more dry rigid electrodes attached to a device box.

The analysis unit is selected from the group consisting of a software on a computer, software on a smartphone, hardware having an Field-Programmable Gate Array (FPGA) architecture, hardware having an Application-Specific Integrated Circuit (ASIC) architecture, and as a standalone unit having software and hardware therein.

The system has communication means for transmitting physiological information through a network to designated recipients for medical evaluation and patient management.

The system employs ECG R-peaks as one means for isolating arterial pulse waves.

The sensing unit comprises dry electrodes.

The analysis unit further comprises a subunit for fusing the oscillometric and puke transit time analyses to obtain robust blood pressure estimation.

The system also has a subunit for evaluating vessel stiffness parameters based on fusing information obtained from the oscillometric and pulse transit time analyses.

The system employs coefficient-based method for evaluating diastolic and systolic blood pressure from oscillometric analysis, comprising steps of: (a) obtaining oscillometric envelope by using ECG R-peak information for de-trending the cuff pressure signal and for isolating arterial pulse waves; (b) using the maximum of the oscillometric envelope for determining mean blood pressure; and (c) using empirical coefficients on the oscillometric envelope for evaluating diastolic and systolic blood pressure.

It also employs a coefficient-based method of evaluating diastolic and systolic blood pressure from pulse transit time analysis, comprising the steps of: (a) calculating pulse transit time between an ECG R-peak and maximum slope on an arterial pulse wave to obtain pulse transit time envelope; (b) using the maximum of the pulse transit time envelope for determining mean blood pressure; and (c) using empirical coefficients on the pulse transit time envelope for evaluating diastolic and systolic blood pressure.

Finally, the system employs a method of evaluating coefficient-free diastolic and systolic blood pressure from pulse transit time analysis, comprising the steps of: (a) calculating pulse transit time between ECG R-peaks and specific points on arterial pulse waves to obtain pulse transit time envelopes; (b) using the maximum of the pulse transit time envelope that is obtained by measuring pulse transit time between ECG R-peaks and bottom of arterial pulse waves, for evaluating diastolic blood pressure; and (c) using the maximum of the pulse transit time envelope that is obtained by measuring pulse transit time between ECG R-peaks and top of arterial puke waves, for evaluating systolic blood pressure.

The system fuses oscillometric and pulse transit analyses to obtain robust blood pressure estimation.

The system has further capability of repeating the evaluation of diastolic and systolic blood pressure periodically, for continuous blood pressure monitoring.

### BRIEF DESCRIPTION OF THE DRAWINGS

A preferred embodiment of the present invention will be disclosed in detail with reference to the drawings, in which:

FIG. 1. shows the ECG-assisted blood pressure monitoring device in use on a human;

FIG. 2 shows the ECG-assisted blood pressure monitoring device;

FIG. 3 shows an operational diagram of the ECG-assisted blood pressure monitoring device;

FIG. 4 shows a circuit diagram of the flexible electrode amplification unit;

FIGS. 5a through 5d show graphical representations of the coefficient-based ECG-assisted oscillometric and the pulse transit time-cuff pressure analyses;

FIGS. 5e and 5f show the oscillometric and pulse transit time envelopes obtained from the coefficient-based ECG-assisted oscillometric and the pulse transit time-cuff pressure analyses;

FIGS. 6a and 6b show a graphical representation of the coefficient-free ECG-assisted pulse transit time-cuff pressure analysis;

FIGS. 6c and 6d show pulse transit time envelopes obtained from the coefficient-free ECG-assisted pulse transit time-cuff pressure analysis; and

FIG. 7 shows a flowchart depicting the method of estimating systolic, diastolic, and mean pressure.

### DETAILED DESCRIPTION OF THE PREFERRED EMBODIMENT

A preferred embodiment of the present invention will be set forth in detail with reference to the drawings, in which like reference numerals refer to like elements or method steps throughout.

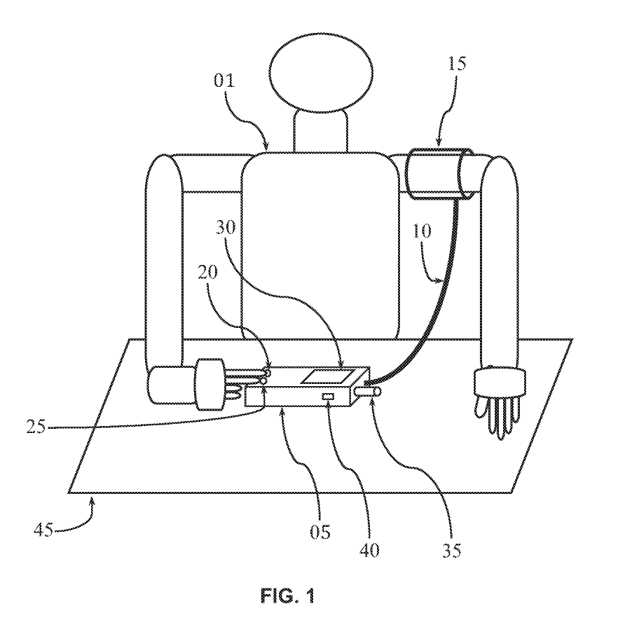

FIG. 1 shows an exemplary system and configuration in which a subject 01 is being monitored by the ECG-assisted blood pressure monitoring device 05 supported on a surface 45. The brachial blood pressure cuff 15, which is worn by the subject 01 on his/her left arm, is connected through an air hose 10 to the device box 05. The active flexible ECG electrodes (not shown) in the blood pressure cuff 15 are connected with wires (not shown), which go through the air hose 10, to the device box 05. In another implementation, wrist blood pressure cuff may be used. The subject 01 touches the active rigid ECG electrode pair 20, 25 attached on the device box 05 with his/her right hand to complete the ECG circuit. The start/stop button 40 is pushed to initiate a recording. Visualization and numerical summary of the physiological parameters monitored are displayed on a liquid crystal display 30 provided on the device box 05. All information is transmitted wirelessly to a personal computer and/or smartphone via the antenna 35. In some embodiments, the device is capable and configured to transmit data wirelessly using a long distance wireless protocol, such as cellular wireless standards, such as GSM, 3G, 4G, or 5G wireless standards. In some embodiments, the device is capable and configured to transmit data wirelessly to communicate with WiFi enabled devices, such as by utilizing the IEEE 802.11 standard for wireless communication. In some embodiments, the device is capable and configured to transmit data wirelessly with other devices under a short range standard, such as the Bluetooth™ standard. One skilled in the art would appreciate that the right side may be used instead of the left for the brachial cuff, and that the rigid electrode pair on the device box should be touched with an opposite limb.

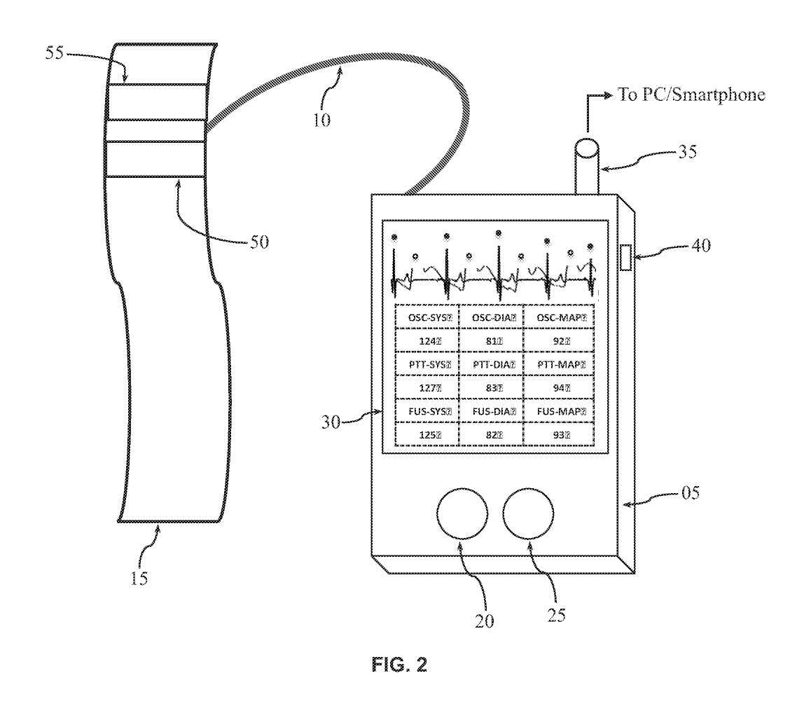

FIG. 2 shows an exemplary close-up of the ECG-assisted blood pressure monitoring device 05. The inner side of the brachial blood pressure cuff 15 is shown along with the active flexible electrode pair 50, 55. In one embodiment these flexible ECG electrodes 50, 55 are rectangular in shape and are made of medical grade silver plated (92% nylon and 8% Dorlastan™) stretchable conductive fabric (0.50 mm thickness and less than 1Ω/Square surface resistivity).

The area of each of these conductive fabric electrodes (50, 55) is about 75 cm². One skilled in the art would appreciate that other electrodes would also perform the invention, for example a flexible electrode can be manufactured from a number of rigid electrodes on a flexible fabric substrate. The electrodes may be dry, gel, or another formulation known in the art, however dry electrodes provide more convenience and less mess. They need not be disposed of as medical waste after each use, as is the case with gel electrodes.

The flexible electrodes 50, 55 are stitched on the inner side of the brachial blood pressure cuff 15, or may be positioned between the cuff and the arm, so as to be held by the cuff 15. Connections to the active flexible electrodes are made using metallic snap buttons (not shown). The air hose 10 connects the bladder (not shown) inside the blood pressure cuff 15 to the device box 05. Wires (not shown), which go through the air hose 10, connect the active conductive fabric electrodes 50 and 55 to the device box 05. In one embodiment, the active rigid ECG electrode pair 20, 25, which are circular in shape, are fixed on top of device box 05. The area of these ECG electrodes (20 and 25) is about 22 cm² each while their thickness is around 4 mm each. A recording is initiated by pushing the start/stop button 40. The liquid crystal display 30 displays all relevant information, for example, mean blood pressure, diastolic blood pressure, systolic blood pressure, etc. In one embodiment, information may be transmitted wirelessly to a personal computer (PC)/smartphone via the antenna 35. In another embodiment (not shown in the FIG. 2) the processing is done locally in the local unit and the information is not transmitted. In yet another embodiment (not shown in the FIG. 2) the information is transmitted using a wired link to a personal computer (PC)/smartphone.

It should be understood that a typical system may include fewer or more electrodes than presented in FIGS. 1 and 2, which may he made and placed in a different way. In preferred embodiment, the sensing electrodes are active. In another embodiment some or all sensing electrodes may be high impedance electrodes. The area of the electrodes and their material may be also different than the ones presented in the preferred embodiment.

The preferred embodiment presented in FIGS. 1 and 2 includes one sensing electrode 50 and one ground electrode 55 under the cuff 15 and one sensing 20 and one ground electrode 25 on the box 05. In another embodiment, zero, one or more ground electrodes may be used.

In one embodiment, the electrodes 20, 25 may be placed on the body and not on the device box. The placement on the body should be such that the heart is in between the electrodes in the cuff 15 and electrodes 20, 25. In another embodiment, all electrodes can be external and not connected to the cuff or to the device box. More than two sensing electrodes may be used. In yet another embodiment, external ECG unit with its electrodes can be connected to the device box or the computer and can be used to acquire analogous ECG data during blood pressure monitoring procedure.

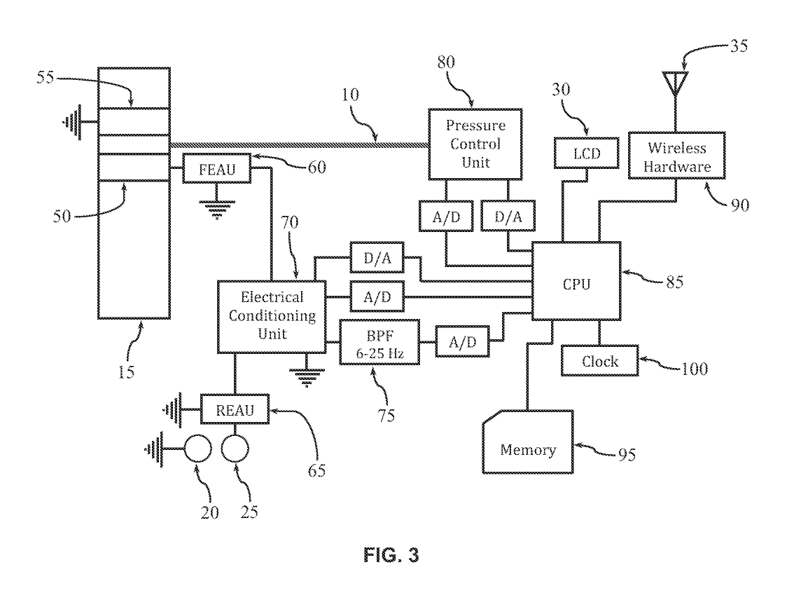

FIG. 3 shows one embodiment of a block diagram of the ECG-assisted blood pressure monitoring device 05 with key components and connections. It should be understood that a typical device may include fewer or more components, connections and configurations. The conductive flexible ECG electrode pair comprises a ground electrode 55 and a sensing electrode 50. The flexible electrode pair 50, 55 connects to the flexible electrode amplification unit (FEAU) 60. The flexible electrode amplification unit 60 is in close proximity to the flexible electrode pair 50, 55—this combination constitutes the active flexible electrode pair on the cuff. Similarly, the rigid ECG electrode pair 20, 25 connects to the rigid electrode amplification unit (REAU) 65—this combination constitutes the active rigid electrode pair on the device box. Both the active flexible electrode pair (50 and 55 along with 60) and the active rigid electrode pair (20 and 25 along with 65) connect to the electrical conditioning unit 70, which has circuitry for further amplification and filtering of the acquired ECG signal. The bladder (not shown) inside the blood pressure cuff 15 connects to the pressure control unit 80 through an air hose 10. The pressure control unit contains a motorized cuff inflation pump, pressure transducer, and a voltage-control pressure release valve (not shown). There are analog to digital (A/D) and digital to analog (D/A) converters between the CPU 85, the electrical conditioning unit 70, and the pressure control unit 80. Moreover, between the CPU 85 and the electrical conditioning unit 70, there is a band-pass filtering (BPF) unit 75 with frequency range 6-25 Hz. Through the band-pass filtering unit 75, the CPU 85 obtains precise and noise-free real-time ECG R-peak information for controlling cuff deflation. The CPU 85 runs software to interact with these modules (70, 75, and 80) to: (i) achieve cuff inflation and (ii) achieve a controlled cuff deflation, during which it acquires simultaneous ECG and oscillometric data. The CPU 85 also runs software for analyzing the acquired ECG and arterial pulse wave data, displaying relevant information on the liquid crystal display 30, storing it in the memory 95, and transmitting it wirelessly to a personal computer/smartphone via the wireless hardware 90 using an antenna 35. The clock 100 attached to the CPU 85 ensures that all information is synchronized and time stamped.

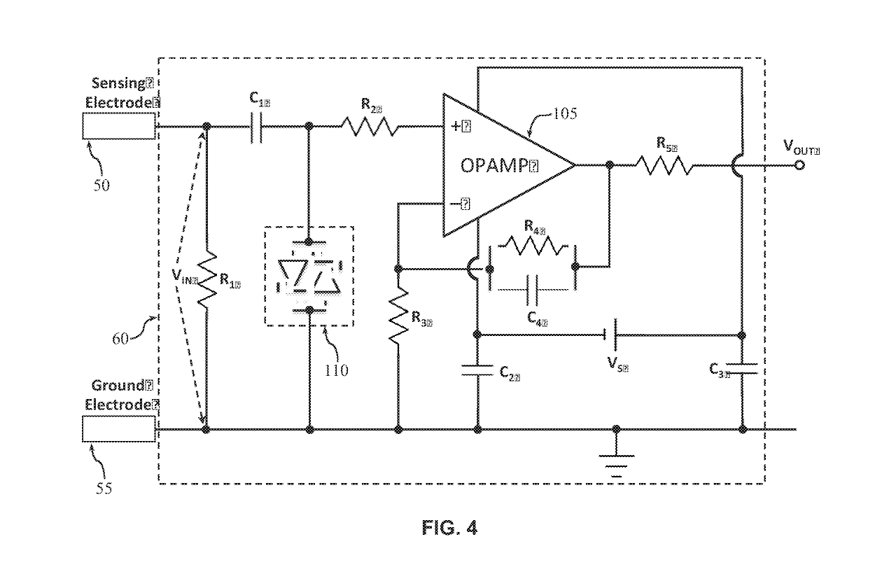

FIG. 4 shows one embodiment of a circuit diagram of the flexible electrode amplification unit 60 for the flexible electrode pair 50, 55. It should be understood that a typical device may include fewer or more components, connections, and configurations. The electrode pair comprises one sensing flexible ECG electrode 50 and one ground flexible electrode 55. The combination of input resistor R₁ and input capacitor C₁, acts as a high-pass filter. This high-pass filter passes all frequencies above 0.1 Hz, thus removing low frequency baseline drift from the ECG signal. For ECG current amplification, a low power operational amplifier (OPAMP) 105 is used. The operational amplifier 105 grounding resistor R₃ and the feedback resistor R₄ provide an ECG voltage gain of (1+R₄/R₃). For example, if R₃=50 KΩ and R₄=250 KΩ, then ECG voltage gain is 6. The combination of operational amplifier 105 feedback resistor R₄ and capacitor C₄, acts as a low-pass filter. This low-pass filter passes all frequencies less than 100 Hz, thus removing high frequency noise from the ECG signal. Therefore, in totality, the flexible electrode amplification unit 60 acts as a band-pass filter with frequency range 0.1-100 Hz. This frequency range is ideal for studying all useful features of an ECG signal. A Schottky diode pair 110 is connected as shown to protect the operational amplifier 105 from static voltage overload and saturation that may occur from the electrode pair side. That is, if V_{IN} exceeds a certain predefined value (for example, if V_{IN}>250 mV), then, the Schottky diode pair 110 will act as a short circuit to discharge current to ground. The capacitors C₂ and C₃ are used for stabilizing the power supply V_{S} to the operational amplifier 105. In this manner, the flexible electrode amplification unit 60, which is in close proximity with the associated electrode pair 50, 55, helps to remove static and other noise to provide good quality amplified output ECG signal V_{OUT}. This amplified ECG signal is then reliably transmitted with minimal transmission loss and noise interference for further amplification, filtering, and digitization. A similar circuit (not shown) is also used for amplifying the ECG signal from the active rigid electrode pair 20, 25.

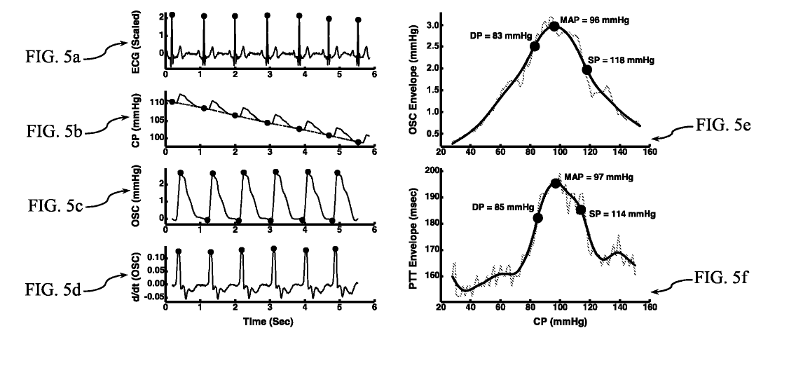

FIGS. 5a through 5d show a six second section of the simultaneous ECG and oscillometric signals acquired by the ECG-assisted blood pressure monitor during cuff deflation and their analysis. FIGS. 5e and 5f show the en Tire oscillometric and pulse transit time envelopes derived from these signals and the estimation of blood pressure from them. The pulse transit time envelope (FIG. 5e) in this case is calculated from ECG R-peak and maximum slope of the arterial pulse wave.

For the coefficient-based ECG-assisted oscillometric analysis, the first step involves the identification of ECG R-peaks, seen as the dots in FIG. 5a. This is followed by superimposing the temporal locations of the ECG ft-peaks on the cuff pressure (CP) signal, the dots in FIG. 5b. A cuff pressure trend line is obtained (dotted line in FIG. 5b) using the ECG R-peak information and is used to de-trend he cuff pressure signal to obtain an oscillometric (OSC) signal (solid line in FIG. 5c). The ECG R-peak Information is also used for finding peaks in the oscillometric signal (upper dots in FIG. 5c)—the maximum amplitude of the oscillometric signal between every two consecutive ECG R-peaks is determined. The oscillmetric puke peak information is used for finding troughs in the oscillometric signal (lower dots in FIG. 5c)—the minimum amplitude of the oscillometric signal between every two consecutive oscillometric pulse peaks is determined. The amplitudes of the oscillometric pulse troughs (lower dots in FIG. 5c) are subtracted from the amplitudes of the oscillometric pulse peaks (upper dots in FIG. 5c), and corresponding cuff pressures (solid line in FIG. 5b) are used to obtain the oscillometric envelope (in FIG. 5e). The maximum of the oscillometric envelope is used for evaluating mean pressure while empirical coefficients are used for evaluating diastolic pressure and systolic pressure (MAP=96 mmHg, DP=83 mmHg, SP=118 mmHg in FIG. 5e).

The coefficient-based pulse transit time-cuff pressure analysis follows from the coefficient-based ECG-assisted oscillometric analysis. First, the oscillometric signal (solid line in FIG. 5c)is differentiated to obtain its derivative (solid line in FIG. 5d). Then the ECG R-peak information (dots in FIG. 5a) is used to find peaks in the derivative of the oscillometric signal (dots in FIG. 5d)—the maximum amplitude of the derivative of the oscillometric signal between every two consecutive ECG R-peaks is determined. Pulse transit time is measured in milliseconds between the ECG R-peaks (dots in FIG. 5a) and the peaks of the derivative of the oscillometric signal (dots in FIG. 5d), and corresponding cuff pressures (solid line in FIG. 5b) are used to obtain the pulse transit time envelope (in FIG. 5f). The maximum of the pulse transit time envelope is used for evaluating mean pressure while empirical coefficients are used for evaluating diastolic pressure and systolic pressure (MAP=97 mmHg, DP=85 mmHg, SP=114 mmHg in FIG. 5f).

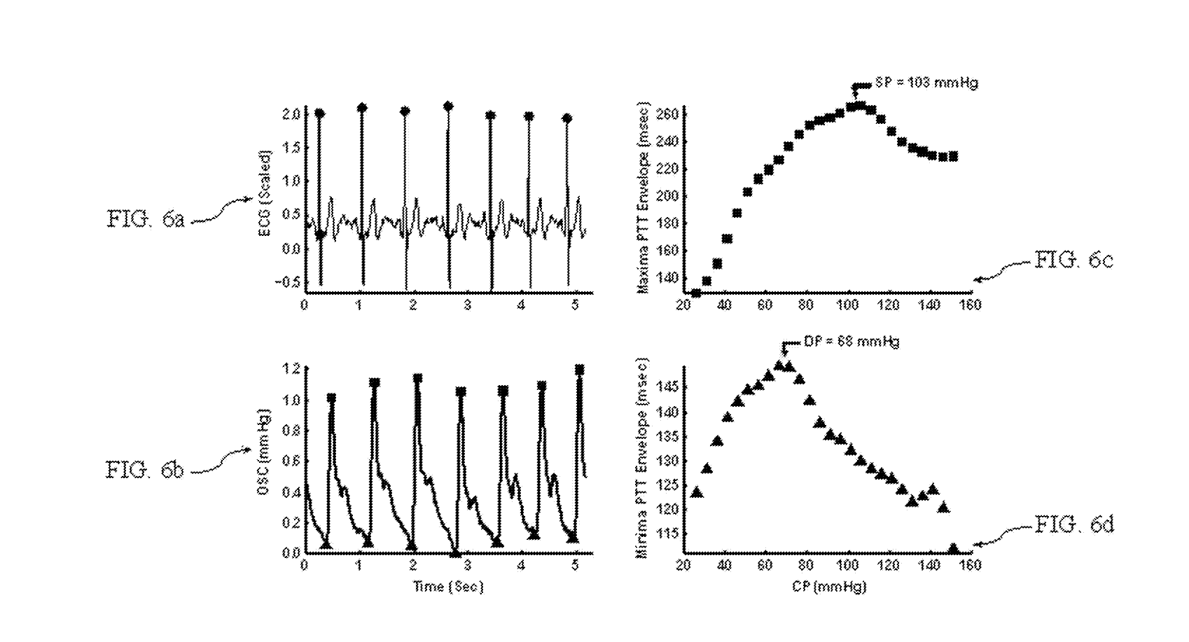

FIGS. 6a and 6b show a five second section of the simultaneous ECG and oscillometric pulse wave signals acquired by the ECG-assisted blood pressure monitor during cuff deflation, and their analysis. FIGS. 6c and 6d show the entire pulse transit time envelopes derived from these signals and the estimation of blood pressure from them.

Pulse transit time is measured in milliseconds between ECG R-peaks (dots in FIG. 6a) and oscillometric (OSC) pulse tops (squares in FIG. 6b), and corresponding cuff pressures (not shown) are used to obtain the maxima pulse transit time envelope (squares in FIG. 6c). The maximum of the maxima pulse transit time envelope is used for evaluating systolic pressure (SP=103 mmHg in FIG. 6c).

Pulse transit time is measured in milliseconds between ECG R-peaks (dots in FIG. 6a) and oscillometric pulse bottoms triangles in FIG. 6b), and corresponding cuff pressures not shown) are used to obtain the minima pulse transit time envelope (triangles in FIG. 6d). The maximum of the minima pulse transit time envelope is used for evaluating diastolic pressure (DP=68 mmHg in FIG. 6d).

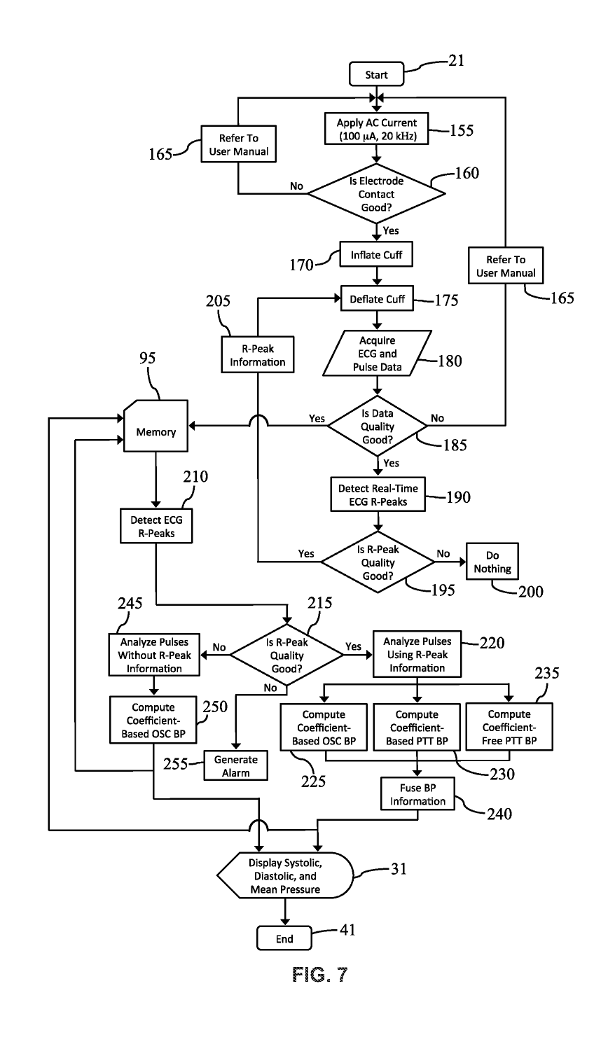

FIG. 7 is a flowchart showing the sequence of steps involved in the operation of the ECG-assisted blood pressure monitor to estimate systolic, diastolic, and mean blood pressure. The analysis unit is divided into a number of subunits for performing different analyses and functions, including steps set forth in FIG. 7 and explained below. When a recording is initiated by pushing the start button at step 21, the device applies alternating current through the electrodes using step 155. Based on the applied alternating current, step 160 checks whether the electrode contact is proper or not. In case the electrode contact is not proper, step 165 advises the user to refer to the user manual for troubleshooting and for reinitiating the measurement. If the electrode contact is proper, then step 170 inflates the cuff to a pressure above the expected systolic pressure. This is followed by initiation of cuff deflation, which is controlled by step 175. During cuff deflation, the analysis unit step 180 acquires simultaneous ECG and oscillometric puke wave data through the electrodes. The quality of the incoming ECG and arterial pulse wave data is checked in real-time by the analysis unit in step 185. If incoming data quality is not satisfactory, step 165 again advises the user to refer to the user manual for troubleshooting and for reinitiating the measurement. If incoming data quality is satisfactory, then the ECG and arterial pulse wave data starts getting stored in the memory at step 95. At the same time, the analysis unit in step 190 starts to detect ECG R-peaks in real-time. Step 195 checks for ECG R-peak quality in real-time. If ECG R-peak quality is not good, then nothing happens (step 200) and the cuff deflation step 175 deflates the cuff without assistance from R-peaks. If ECG R-peak quality is satisfactory, then step 205 feeds ECG R-peak information to the cuff deflation step 175, which is then controlled by R-peaks. Once the cuff is deflated below the expected diastolic pressure, the measurement is complete.

In FIG. 7, that analysis unit at step 210 analyzes ECG data stored in memory step 95 to detect R-peaks. Step 215 checks the quality of R-peaks. If ECG R-peak quality is not satisfactory, then step 245 analyzes arterial pulses without assistance from R-peaks. At step 250 th analysis unit then creates an oscillometric (OSC) envelope and computes blood pressure using empirical coefficients. This information, which comprises systolic, diastolic, and mean pressure, is then presented to the user through the display step 31. Moreover, If ECG R-peak quality is not satisfactory and blood pressure is computed without ECG R-peak assistance, at step 255 the analysis unit generates an alarm to alert the user. The user can then push the end button at step 41 to stop the monitor.

In FIG. 7, if the analysis unit at step 215 determines the ECG R-peak quality to be satisfactory, then at step 220 analyzes arterial pulses with the assistance of R-peaks. At step 225 it creates an R-peak assisted oscillometric envelope to estimate blood pressure using empirical coefficients. At step 230 the analysis unit creates a pulse transit time (PTT) envelope (by measuring time between ECG R-peak and maximum slope of arterial pulse peak) to estimate blood pressure using empirical coefficients. At step 235 the analysis unit creates two pulse transit time envelopes (one by measuring time between ECG R-peak and top of arterial pulse peak and other by measuring time between ECG R-peak and bottom of arterial pulse peak) to estimate blood pressure without empirical coefficients. The information from the three blood pressure estimations at steps 225, 230, and 235 is then sent to the blood pressure information fusion at step 240, which optimizes and fuses this information to generate a single estimate of systolic, diastolic, and mean pressure. This information is then presented to the user through the display step 31. The user can then push the end button at step 41 to stop the monitor.

The embodiments presented will allow users to acquire ECG signal during regular, almost unchanged, blood pressure monitoring routine. They also allow for an alternative way of estimating systolic and diastolic blood pressure, which is more robust especially in cases of obesity, arrhythmias and atrial fibrillation. In addition, vessel stiffness is estimated. While above description contains many specificities, these should not be construed as limitations on the scope of the invention, but rather as an exemplification of preferred embodiments thereof. Many other variations are possible. For example, the blood pressure monitor can present other physiological parameters extracted from the ECG signal, for example, heart rate variability metrics. The monitor can be used as a wearable blood pressure monitor where ECG and blood pressure can be acquired periodically for long-term blood pressure monitoring. The blood pressure monitor can be integrated with a smartphone in a way that it is physically attached to the smartphone and can be used as a single device in which case all the processing will be done directly on the smartphone.

Accordingly, the scope of the invention should be determined not by the embodiments illustrated, but by the appended claims and their legal equivalents.

The invention claimed is:

1. A sensing unit, comprising
a. a cuff for measuring blood pressure, comprising a first dry flexible sensing electrode positioned between a human body part and an inside surface of the cuff, for connection to a human body;
b. one or more dry flexible ground electrodes positioned between a human body part and an inside surface of the cuff for connection to the human body; and
c. a second sensing dry electrode for connection to the human body such that a heart of the human body is intermediate the first sensing and second sensing electrodes.

2. The system of claim 1, further comprising a second dry ground electrode near the second sensing electrode, for equalizing static potential on body and reducing noise.

3. The system of claim 1, wherein the first and second sensing electrodes are active electrodes to reduce transmission noise.

4. The system of claim 1, wherein the first and second sensing electrodes are high impedance electrodes to reduce half-cell potential.

5. The system of claim 1, further comprising a device box, wherein the second sensing electrode is positioned on the device box.

6. A system for non-invasive blood pressure estimation comprising:
a. an electrocardiogram (ECG) measuring unit;
b. an arterial pulse wave measuring unit in communication with the ECG measuring unit;
c. a cuff for measuring blood pressure in communication with the arterial pulse wave measuring unit;
d. two or more electrodes connected to the ECG measuring unit;
e. an analysis unit connected to the ECG and arterial puke wave measuring unit, the analysis unit comprising,
i. an ECG measuring subunit;
ii. a cuff pressure and arterial puke wave measuring subunit;
iii. a subunit that uses ECG R-peak information for de-trending cuff pressure signal;
iv. a subunit that uses ECG R-peak information for isolating arterial pulse waves;
v. a subunit for measuring pulse transit time between ECG R-peak and specific points on the arterial pulse wave and mapping the measured pulse transit time with corresponding cuff pressure, obtaining pulse transit time envelopes;
vi. a subunit for de-trending cuff pressure signal and finding arterial pulse wave amplitude using ECG R-peak information, and mapping the measured amplitude with corresponding cuff pressure, obtaining an oscillometric envelope;
vii. a subunit for analyzing morphology of pulse transit time envelopes, to obtain pulse transit time-based blood pressure estimation; and
viii. a subunit for analyzing morphology of the oscillometric envelope, to obtain oscillometric blood pressure estimation.

7. The system of claim 6, wherein the ECG measuring unit uses a high frequency, low magnitude current for checking the quality of contact between the electrodes and the human body.

8. The system of claim 6, wherein the ECG measuring unit comprises one or more flexible dry electrodes attached to the internal surface of a brachial cuff and one or more dry rigid electrodes attached to a device box.

9. The system of claim 6, wherein the analysis unit is selected from the group consisting of a software on a computer, software on a smartphone, hardware having an Field-Programmable Gate Array (FPGA) architecture, hardware having an Application-Specific Integrated Circuit (ASIC) architecture, and as a standalone unit having software and hardware therein.

10. The system of claim 6, further comprising communication means for transmitting physiological information through a network to designated recipients for medical evaluation and patient management.

11. The system of claim 6 wherein ECG R-peaks are used as one means for isolating arterial pulse waves.

12. The system of claim 6, wherein the electrodes are dry electrodes.

13. The system of claim 6, wherein the analysis unit further comprises:
ix. a subunit for fusing the oscillometric and pulse transit time analyses to obtain robust blood pressure estimation.

14. The system of claims 6, wherein the analysis unit further comprises:
ix. a subunit for evaluating vessel stiffness parameters based on fusing information obtained from the oscillometric and pulse transit time analyses.

15. The system of claim 6, further comprising a subunit that employs a coefficient-based method for evaluating diastolic and systolic blood pressure from oscillometric analysis, comprising steps of:
a. obtaining oscillometric envelope by using ECG R-peak information for de-trending the cuff pressure signal and for isolating arterial pulse waves;
b. using the maximum of the oscillometric envelope for determining mean blood pressure; and
c. using empirical coefficients on the oscillometric envelope for evaluating diastolic and systolic blood pressure.

16. The system of claim 6, further comprising a subunit that employs a coefficient-based method of evaluating diastolic and systolic blood pressure from pulse transit time analysis, comprising the steps of:
a. calculating pulse transit time between an ECG R-peak and maximum slope on an arterial pulse wave to obtain pulse transit time envelope;
b. using the maximum of the pulse transit time envelope for determining mean blood pressure; and
c. using empirical coefficients on the pulse transit time envelope for evaluating diastolic and systolic blood pressure.

17. A method of evaluating diastolic and systolic blood pressure from pulse transit time analysis, comprising the steps of:
a. calculating pulse transit time between ECG R-peaks and specific points on arterial pulse waves to obtain pulse transit time envelopes;
b. using the maximum of the pulse transit time envelope that is obtained by measuring pulse transit time between ECG R-peaks and bottom of arterial pulse waves, for evaluating diastolic blood pressure; and
c. using the maximum of the pulse transit time envelope that is obtained by measuring pulse transit time between ECG R-peaks and top of arterial pulse waves, for evaluating systolic blood pressure.

18. The method of claim 17, further comprising the step of fusing the oscillometric and pulse transit analyses to obtain robust blood pressure estimation.

19. The methods of claim 17, further comprising the step of repeating the evaluation of diastolic and systolic blood pressure periodically, for continuous blood pressure monitoring.
